(19) United States
(12) Patent Application Publication
(10) Pub. No.: US 2026/0276519 A1
(43) Pub. Date: Sep. 17, 2026

(54) SPATIALLY RESOLVED MEMBRANE MOLECULE ANALYSIS

(71) Applicants: Dimitrios STAMOU, Charlottenlund (DK); Gabriele KOCKELKOREN, San Francisco, CA (US); Line LAURITSEN, Odense M (DK); Christopher SHUTTLE, Helsinki (FI); Artu BREUER, Copenhagen (DK); Eieftheria KAZEPIDOU, Copenhagen (DK)

(72) Inventors: Dimitrios STAMOU, Charlottenlund (DK); Gabriele KOCKELKOREN, San Francisco, CA (US); Line LAURITSEN, Odense M (DK); Christopher SHUTTLE, Helsinki (FI); Artu BREUER, Copenhagen (DK); Eieftheria KAZEPIDOU, Copenhagen (DK)

(21) Appl. No.: 19/469,549

(22) PCT Filed: Mar. 29, 2024
(86) PCT No.: PCT/EP2024/058817

§ 371 Date: Sep. 26, 2025

(30) Foreign Application Priority Data
Mar. 29, 2023 (EP) 23165274.4

(51) Int. Cl. G01N 15/1429; G01N 15/10; G01N 15/14; G01N 15/1434; G01N 33/53; G01N 33/68

(52) U.S. Cl. CPC G01N 15/1431; G01N 15/1434; G01N 15/1468; G01N 33/53; G01N 33/68; G01N 2015/1006; G01N 2015/144; G01N 2333/705

(57) ABSTRACT

The present invention suggests a method and a system for characterizing a membrane bound molecule. The method comprises determining a plurality of spatially resolved membrane properties of a biological membrane, determining, for a plurality of locations, a spatially resolved membrane molecule signal related to the membrane bound molecule at the plurality of locations on the membrane and grouping the spatially resolved membrane molecule signals to classes of at least one of the membrane properties or state classes of the membrane bound molecules. The method further comprises identifying the distribution of conformational states of the membrane bound molecule based on the distribution of the classes.

The present invention relates to methods, apparatuses and systems for analysing biological molecules and their pharmacological parameters in cell membranes. In particular, the present disclosure relates methods, systems, and apparatuses for fluorescence imaging of conformational states and changes in the conformational states of membrane bound molecules.

Many biological molecules can occur in different conformational states and can change the conformational state upon external triggers. These conformational states and conformational changes underpin the function of biological molecules and are thus important information for many biological and pharmacological processes. For example, many methods were developed to determine ligand binding to receptor proteins and/or conformational changes of proteins in cell membranes. Among others, fluorescence methods and techniques are used today to determine the efficacy of ligand binding and to monitor conformational changes in the biomolecules.

Most methods used today determine conformational changes in bulk thus averaging over many molecules in a single cell and typically many cells in a cell culture. Some methods have been developed to analyse ligand binding or conformational changes on a single molecule level but most industrial applications measure ligand binding and conformational changes of biomolecules in bulk.

Membrane molecules are complex biological systems and there are many additional effects that cannot be revealed by the methods available today.

Recently developed methods have shown that membrane molecules form clusters in specific areas of cell membranes.

There is a need for improved apparatuses and methods for measuring conformational states and/or conformational changes of biomolecules.

### SUMMARY OF THE INVENTION

The present invention suggests methods and systems for characterizing a membrane bound molecule.

In one aspect, the present invention relates to a method for characterizing a distribution of states of a membrane bound molecule. The method comprises determining a plurality of spatially resolved membrane properties of a biological membrane, determining, for a plurality of locations, a spatially resolved membrane molecule signal related to the membrane bound molecule at the plurality of locations, and grouping the spatially resolved membrane molecule signals to classes of at least one of the membrane properties or state classes of the membrane bound molecules. The method further comprises identifying the distribution of states of the membrane bound molecule based on the distribution of the classes. The states of the membrane bound molecule may correspond to conformational states of the membrane bound molecule.

In another aspect, the present invention relates to a method for characterizing a state of a membrane bound molecule. The method comprises determining a plurality of spatially resolved membrane properties of a biological membrane, determining, for a plurality of locations, a spatially resolved membrane molecule signal related to the membrane bound molecule at the plurality of locations on the membrane and grouping the spatially resolved membrane molecule signals to classes of the membrane properties to obtain a first state specific membrane molecule pattern. The method further comprises inducing a conformational change to the membrane bound molecule, and monitoring a change in the grouping of the spatially resolved receptor signals and obtaining a second state specific membrane molecule pattern.

In yet another aspect, the present invention discloses a method for characterizing a state of a membrane bound molecule. The method comprises determining a plurality of spatially resolved membrane properties of a biological membrane, determining, for a plurality of locations, a spatially resolved membrane molecule signal related to the membrane bound molecule at the plurality of locations on the membrane, and grouping the spatially resolved membrane molecule signals to classes of the membrane properties to obtain a state specific membrane molecule pattern. The method further comprises comparing the state specific membrane molecule pattern with patterns in a database and identifying a conformational state of the membrane bound molecule.

The methods are alternative solutions that relate to the identification and characterization of state specific patterns on one end and to the identification of specific states or conformational states based on the pre-characterized state specific patterns.

The present disclosure also suggests an apparatus and a system for carrying out the methods of the present disclosure.

### DESCRIPTION OF THE FIGURES

The invention may be better understood when reading the detailed description of examples of the present disclosure which is given with respect to the accompanying figures in which:

FIG. 1 shows the generation of high-accuracy topography maps of plasma membranes of living cells,

FIG. 2 shows an example of how beta1 AR density and activation is organized into domains at the plasma membrane,

FIG. 3 shows an example of domain-based characterization of different beta1 AR ligands and different concentrations of the same ligand,

FIG. 4 shows examples of conformational states of GLP1R are segregated by plasma membrane curvature,

FIG. 5 shows examples of how different ligands give rise to different patterns of beta1 AR activation in curvature space,

FIG. 6 shows three distinct spatial populations revealing existence of four beta1AR activity states, and

FIG. 7 show the redistribution of beta1 AR activation probabilities and beta1 AR states by full and inverse agonists.

### DETAILED DESCRIPTION

Examples of the present disclosure will now be described in more detail. It is to be understood that the described examples and the examples shown in the figures are purely illustrative for the methods described. The methods of the present disclosure are not limited to specific biological systems and a person skilled in the art will amend the examples according to specific requirements. It is not necessary to implement all features shown in the examples and a person skilled in the art will combine features shown with respect to one figure with examples shown in other figures.

The present disclosure relates to methods, systems and apparatus' for characterizing or determining at least one parameter and/or states of membrane molecules. An example for a parameter of membrane molecules is the distribution of conformational states of membrane molecules. Membrane molecules are molecules that are linked to, attached to or integrated in a membrane and are termed membrane bound molecules herein. A group of membrane bound molecules of interest for medical and pharmacological applications are membrane proteins such as membrane bound proteins that are attached to a biological membrane or transmembrane proteins that extend through a biological membrane.

Membranes are biological membranes made from a plurality of lipid molecules in the examples. Biological membranes are typically lipid bilayers and may be termed lipid membranes. Examples for the biological membrane are cell membranes, in particular the plasma membrane of a cell. Membrane molecules also comprise lipids or groups of lipids. So further examples refer to biological membranes. However, the concept of the present disclosure may be applied to other membranes systems in a similar manner.

Among membrane proteins, ligand receptors are a group of membrane bound proteins that are of pharmacological interest. Examples of the present disclosure were carried with G-protein coupled receptors (GPCRs) and receptor tyrosine kinases as examples systems. The method of the present disclosure has been tested with these systems but the methods are generic. It is evident that the methods disclosed herein are not limited by the specific receptors used in the examples. The methods can be applied with other receptors or other membrane molecules in a corresponding way. Examples for other membrane molecules comprise, but are not limited to ion channels, enzymes or transporters.

In a first aspect, the method comprises determining a plurality of spatially resolved membrane properties of the biological membrane, determining, for a plurality of locations, a spatially resolved membrane molecule signal related to the membrane bound molecule at the plurality of locations on the membrane, and grouping the spatially resolved membrane molecule signals to classes of the membrane properties to obtain a first state specific membrane molecule pattern.

Determining the plurality of spatially resolved membrane properties comprises determining the membrane property in different areas of the membrane. In other words, determining the plurality of spatially resolved membrane properties comprises determining membrane property values representative for the membrane property in different distinct membrane areas. A membrane area can be a certain area or section on the membrane, like a pixel. The membrane area may also be a voxel, as the membrane has a three dimensional position in space. The membrane properties may be recorded in two-dimensional matrix and/or may be plotted in a two-dimensional map.

The size of the membrane area, and such the spatial resolution of the spatially resolved membrane properties can be chosen depending on the membrane and the technology used. If fluorescent labels are used, a resolution of 250 nm has a sufficient disclosure. 10 nm or better can be achieved. An area can correspond to the resolution of the measurement set-up or can be chosen differently.

The membrane properties may be a topography, a curvature, a tension or a fluidity of the membrane or any combination thereof. In another example the membrane property may be a presence or absence of specific membrane components. In yet another example the membrane property may be the density of a membrane molecule, for example the density of membrane lipid molecules and/or the lipid composition of the membrane or the density of molecules bound in or on the membrane. Other membrane properties or a combination of membrane properties may also be used. The membrane property may be an average or mean value in each of the areas, pixels or voxels. The membrane property may be determined in a spatially resolved manner by microscopy. Microscopy may be implemented using light, for example transmitted, reflected, fluorescent or bioluminescent, or electrons. Other imaging mechanisms or principles may also be used to acquire microscopy images.

Determining, for a plurality of locations, a spatially resolved membrane molecule signal related to the membrane bound molecule comprises determining a spatial position of the signal related to the membrane. Spatial resolution of the density may vary on the requirements and may be adapted to the biological system under investigation. It may correspond to the spatial resolution of the membrane properties but a different resolution may be used as well.

Determining, for a plurality of locations, a spatially resolved membrane molecule signal may comprise determining a state specific spatially resolved membrane molecule signal. The signal relates to a state of the membrane bound molecule. Biological molecules mostly co-exist in different states at the same. The distribution of the co-existing states depends on the external conditions and comes to an equilibrium of states after some time. This distribution of the co-existing states is determined in a spatially resolved manner, i.e. in a plurality of locations on the membrane. With a change of external conditions, the distribution among the different states changes. This change may be different in different locations and may be determined in a spatially resolved manner.

Determining, for a plurality of locations, a spatially resolved membrane molecule signal may comprise fluorescent imaging of fluorescent labels that can represent the presence of the membrane molecule. For example, the fluorescent label may be attached to the membrane molecule. Examples for fluorescent labels include, but are not limited to, fluorescent proteins or organic dyes.

Fluorescent imaging may comprise laser scanning confocal microscopy. Alternative methods are wide field, TIRF, spinning disk confocal, lightsheet, STED, PALM, STORM and other methods that allow to determine a density of fluorescent signals on the biological membrane.

Classes, categories of groups of membrane properties may be defined. For example, a multi-dimensional matrix and/or map of the membrane properties may be determined to provide a spatially resolved map in the dimensions of the membrane properties. The map may be two dimensional or may comprise more dimensions. Areas of the membrane may be categorized according to the membrane properties. For example, membrane areas with a membrane mean curvature larger than a pre-determined value may be classified as area with high mean curvature and other areas may be classified as areas with low mean curvature. Similar areas can be classified depending in the two principal curvatures, lipid density, protein density or on any other membrane property of interest. In some examples domains where a membrane property exceeds a pre-determined value may be identified.

The membrane properties may have a local influence on the behaviour or characteristics of the membrane protein. The protein signal may be different in areas of higher membrane curvature compared to areas that have no or only a small membrane curvature. Other examples revealed, that the protein signal varies depending on at least one of a presence of specific lipids in the membrane, a lipid composition, a density of lipids.

The spatially resolved membrane molecule signals are grouped into classes.

The spatially resolved membrane molecule signals can be grouped into classes of at least one of the membrane properties or state classes of the membrane bound molecules. The sate classes may be different states the membrane bound molecule can take. The spatially resolved membrane molecule signals may form domains and the domains may be related to classes of categories. The domains may be defined where a molecule-state or signal, for example where a receptor to sensor ratio, exceeds or falls below a threshold.

The spatially resolved membrane molecule signals are grouped into the classes of the membrane properties to obtain a first state specific membrane molecule pattern. The classes of membrane properties are described above. For example, the signals may be grouped in a membrane curvature space resolving the membrane principal curvatures C1 and C2 in two dimensions. Besides membrane curvature space, a lipid density space or a lipid composition space may be used.

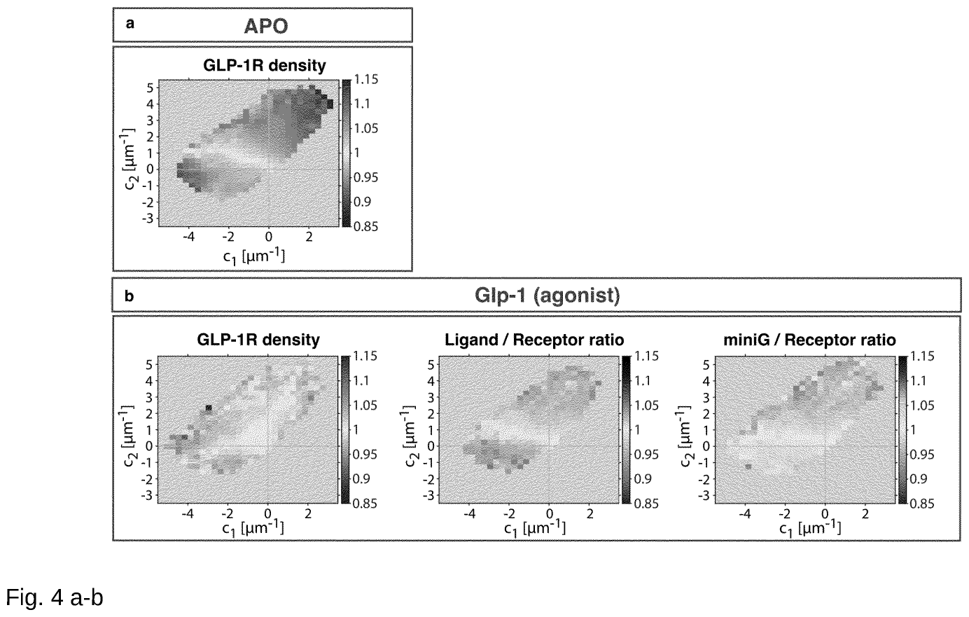

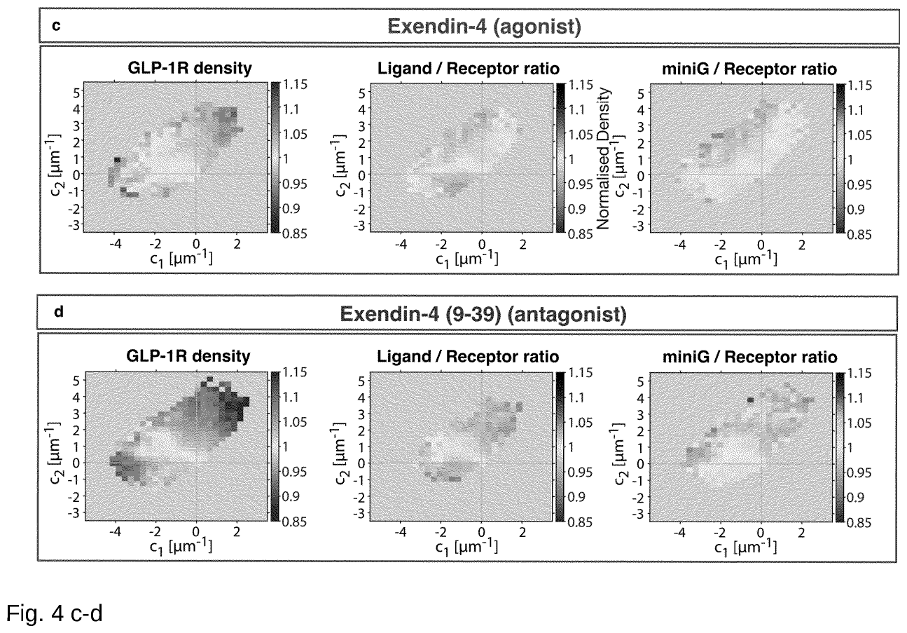

The grouping of the spatially resolved membrane molecule signals can result in a specific pattern that is specific for a membrane molecule in a particular state and a particular membrane. The pattern may be a one-, two-, or multidimensional representation of the grouped or classified membrane molecules signals. The pattern may have domains, maxima, minima and other features that a representative for a graph or map. An example for such a pattern in the curvature space is shown in FIG. 4a. Other examples may be in a topography space, for example in an XY space, an XYZ space. Furter examples are a lipid density space or lipid composition space or combinations thereof. The representation may then be one dimensional or may be multidimensional in case a multi-lipid composition. The particular state may correspond to a conformational state of the membrane molecule.

The inventors found that this pattern changes in dependence of the environment of the membrane molecule and/or in dependence of a conformational state of the membrane molecule. For example, the pattern changes if ligands are added to the receptors. Moreover, the pattern changes specifically depending on the ligand, on the type of ligand and on the ligand concentration. Thus, a first pattern corresponds to a first conformational change and, a second pattern corresponds to a second conformational change.

In one aspect, this allows to identify specific patterns, that are representative for a conformational state of the membrane molecule. Thus, the method comprises one aspect to induce a conformational change to the membrane bound molecule, and monitoring a change in the grouping or distribution of classes of the spatially resolved receptor signals and obtaining a second state specific membrane molecule pattern. Thus, the second state specific membrane molecule pattern is specific to the induced change. For example, the addition of a particular ligand at a particular concentration will induce a pattern that is specific for this situation. The induced conformational change, can be the addition of ligands, agonist, antagonist. It can also be another environmental change, such as temperature, pH-value or others.

In another example, classes, categories or groups of states of the membrane bound molecules may be defined. Grouping into the classes is done according to different states of the membrane bound molecules. The sates can be, for example, conformational states or can relate to the conformational states or can be other states of the membrane bound molecule. This allows identifying the distribution of different conformational states of the membrane bound molecule base on the distribution of classes.

The methods allow to determine and characterize changes of the distribution of conformational states or of conformational changes of the membrane molecule itself or along a signal pathway in dependence of the molecule density at or in the biological membrane.

In some examples, the determining the spatially resolved membrane molecule signal comprises determining at least one of a ratio of signals of an antibody to membrane bound molecule, ligand to antibody, ligand to membrane bound molecule, a conformational biosensor to membrane bound molecule, antibody to conformational biosensor, and ligand to conformational biosensor. This provides more detailed insight in the conformation states of some of the membrane molecules. A conformational biosensor is a molecule that reports the presence of a specific conformation.

In a second aspect, the specific patterns, distributions of states and/or matrices may be registered in a look-up table. This look-up table can be a database. The database may then contain a plurality of patterns or distribution patterns that are specific and representative for a state of the membrane molecules. A pattern or distribution measured as described above can then be compared to patterns from the look-up table to identify a conformational state of the membrane molecule.

In yet other aspects, the method comprises determining a spatial molecule density related to a density of the membrane molecule for a plurality of locations on the biological membrane. The method further comprises determining a conformational change of the membrane protein related to at least one of the plurality of locations, and correlating the conformational change to the spatial molecule density of the membrane molecule.

In one example, fluorescence may be used for determining the conformational change relating to the membrane molecule. Fluorescence methods with different fluorescent tags can be used with substantially the same measurements system, where for example different wavelength for different fluorescent tags are used. However, other ways to determine conformational changes have been developed in recent years that can be used with the present disclosure as well requiring additional measurement set-up.

A second type of fluorescent labels may be attached to the molecule or in the signalling pathway of the membrane molecule. The fluorescence properties may alter in dependence of the conformational change and such a system may be described as a conformational biosensor. Here, the conformational biosensor is a molecule that reports the presence of a specific conformation as a fluorescence readout. For example, the conformational biosensor is a molecule that recognises and binds specifically to one conformation, such as antibodies or the truncated G-proteins. Specific examples for the systems described in the examples are FPs permutatated into the GPCR sequence that fold and change fluorescence as the receptors undergo conformational changes.

Correlating the conformational change to the spatial molecule density of the membrane molecule comprises in one example evaluating conformational changes in dependence of the molecule density. The inventors have found the conformational changes occur differently depending on the molecule density. In an example, membrane molecules are grouped in two or more classes of densities and the conformational changes are determined for each of the classes. For example, the response to ligand binding, may be different depending on the molecule density. Density dependent ligand binding or ligand efficacy may be determined in one example. In another example, the change of the conformational state of membrane molecule, for example a membrane protein may depend on the density of the membrane molecules. One state may be favourable over a second state in areas with higher or lower membrane molecule density.

3D measurements reveal information of receptor activation and localization in real space (x, y and z) and in curvature space. This allows for the pharmacological characterization of ligands in both parameter spaces.

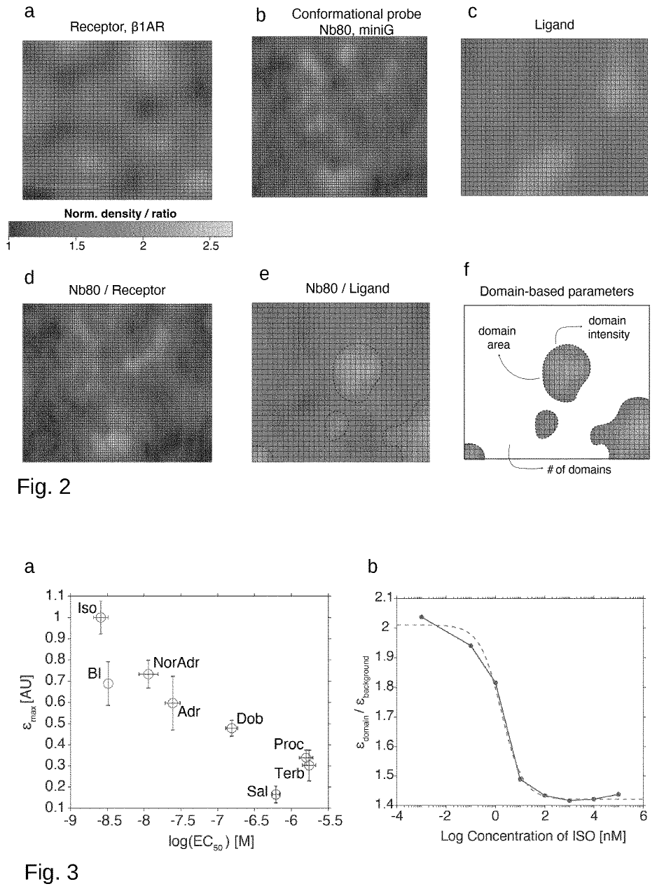

The methods and systems allow to obtain the relative density of receptors, conformational probes, and ligands (See, FIG. 2a-c as examples) mapped onto the 3D surface of the plasma membrane of living cells. The inventors found that these three key signalling components are spatially organized into domains.

These local density measurements are used to gain understanding of spatially resolved receptor signalling. In one aspect, the ratio of the conformational probe and the receptor density are mapped as a measure for the relative activation probability (Seen FIG. 2d as an example). Second, the intrinsic efficacy of ligands can be spatially resolved by mapping the ratio of the conformational probe to ligand density (Exemplary results shown in in FIG. 2e).

The probability for a ligand to activate a receptor and, subsequently stabilize the conformation is organized into domains at the plasma membrane as can be seen from the examples of FIGS. 2d and 2e. By applying a thresholding algorithm to isolate individual domains, a ligand can be characterized by its intrinsic efficacy, by the area of individual domains and by the number of domains it creates (FIG. 2f). This methodology can be employed to characterize any ligand for any ligand concentration of interest and is not limited to the examples shown and described.

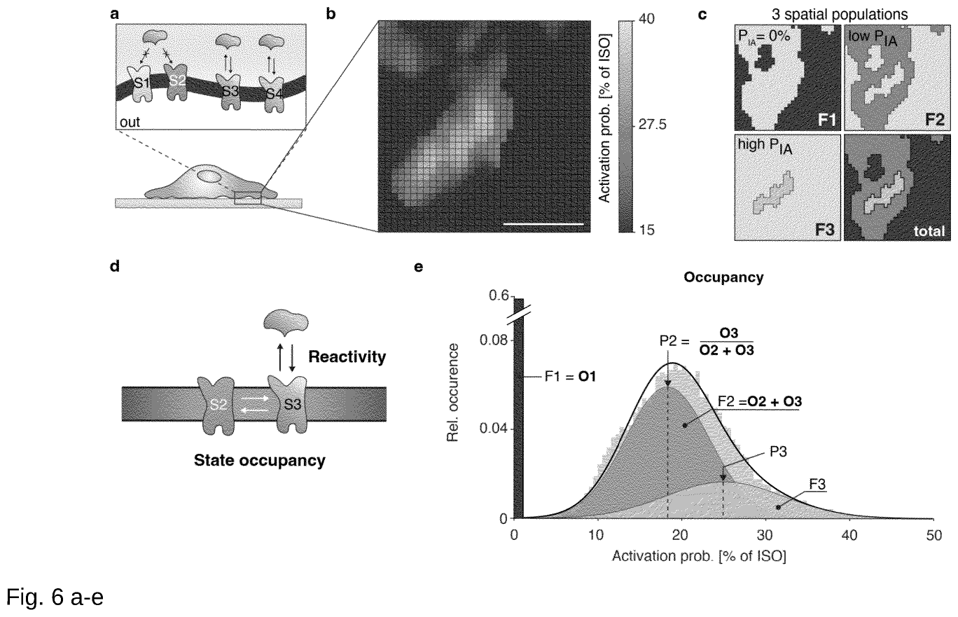

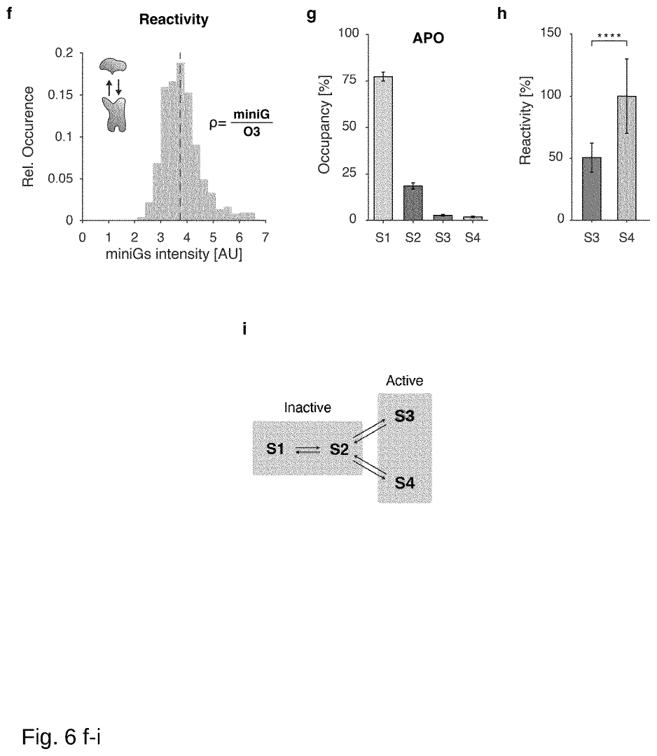

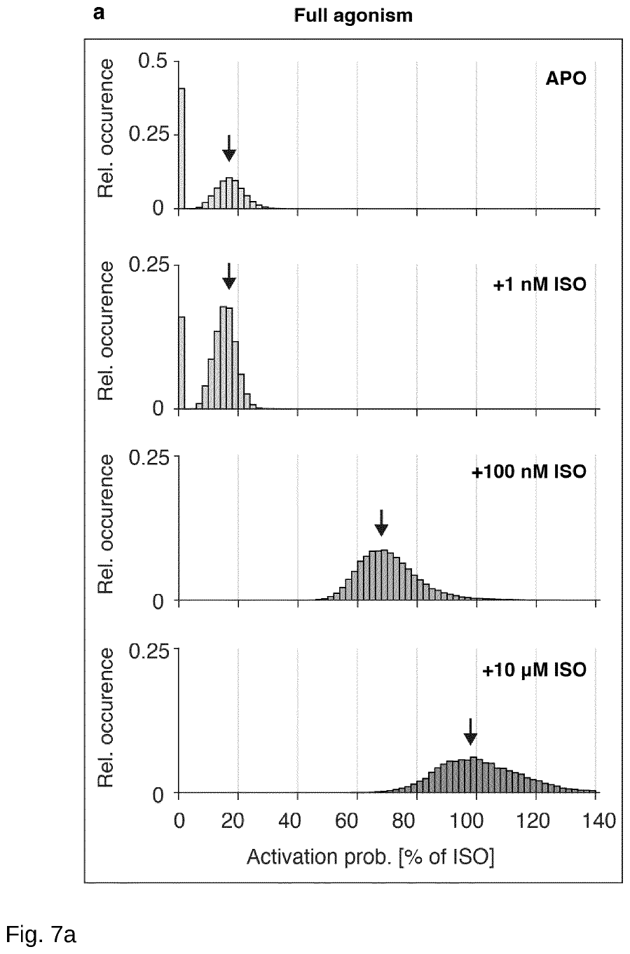

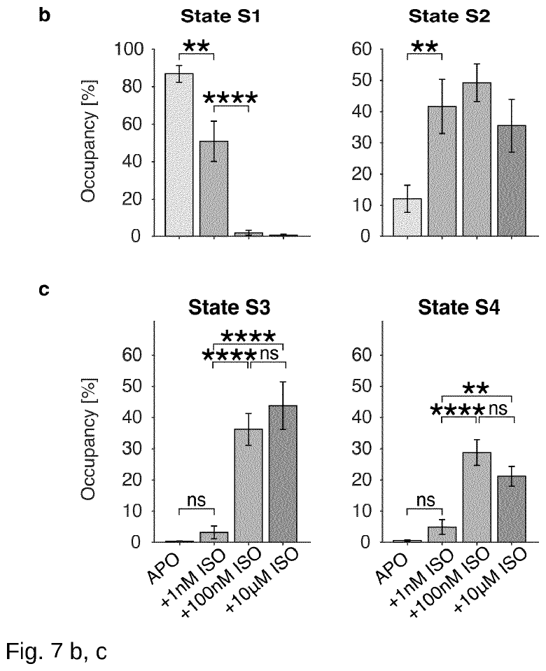

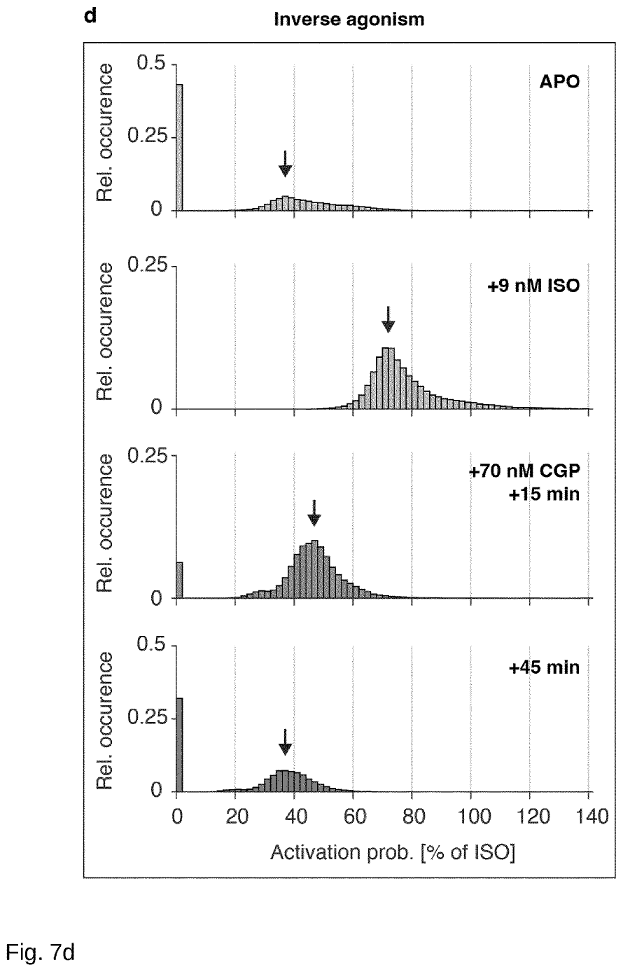

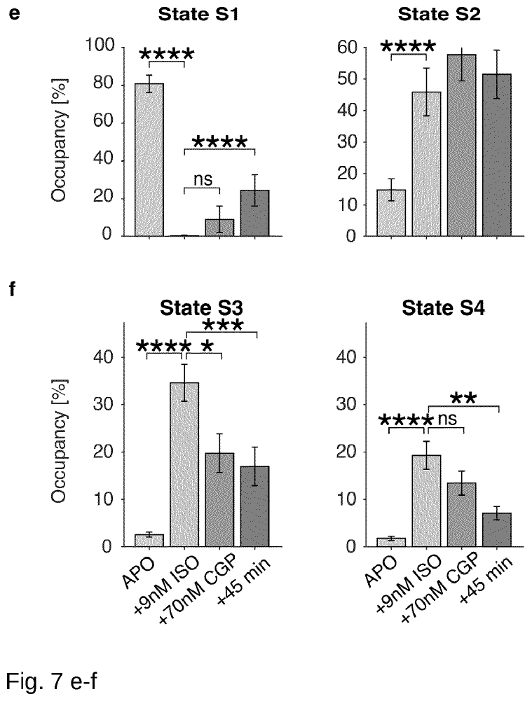

Biological molecules mostly co-exist in different states. With a change of external conditions, the distribution among the different states changes. For example, signaling efficacy and selectivity of GPCR-drugs stem from their ability to modify the distribution among four primary conformational states. Spatial patterns of GPCR intrinsic activation at the plasma membrane and corresponding results are shown in FIGS. 6 and 7.

The methods and systems described herein have been evaluated and tested in several experiments using several exemplary systems. Some examples are described below. It is obvious to a person skilled in the art that the methods, systems, and corresponding apparatuses are not limited to these biological examples and that the method and systems, and apparatuses can be used with other membrane bound systems.

### EXAMPLES

### Live-Cell Microscopy

Imaging was performed on an Abberior Expert Line system with an Olympus IX83 microscope (Abberior Instruments GmbH). For imaging SNAP-Surface649 and eGFP we used respectively 640 nm or 488 nm pulsed excitation laser; fluorescence was detected between 650-720 nm or 500-550 nm, respectively. For imaging mRuby2 and HALO-JF549 we used 561 nm pulsed excitation and fluorescence was detected between 580-630 nm. Cross-excitation was avoided by sequential imaging. All XZY stacks were recorded by piezostage (P-736 Pinano, Physik Instrumente, Germany) scanning using a voxel size of 30 nm (dx=dy=dz=30 nm). We used a UPlanSApo ×100/1.40 oil immersion objective lens and a pinhole size of 1.0 Airy units (i.e., 100 [symbol] m). Alignment of confocal channels was adjusted and verified on Abberior auto-alignment sample. All measurements were made at room temperature and acquired in confocal imaging mode.

### Data Analysis

The 3D imaging method of the present disclosure simultaneously, but independently, recovers 1) high-accuracy membrane topography and curvature and 2) protein density of any membrane-associated protein of interest. In the sections below, these two key principles are described in detail and considerations in method development are outlined.

Reconstructing High-Accuracy Topography Maps with Confocal Microscopy

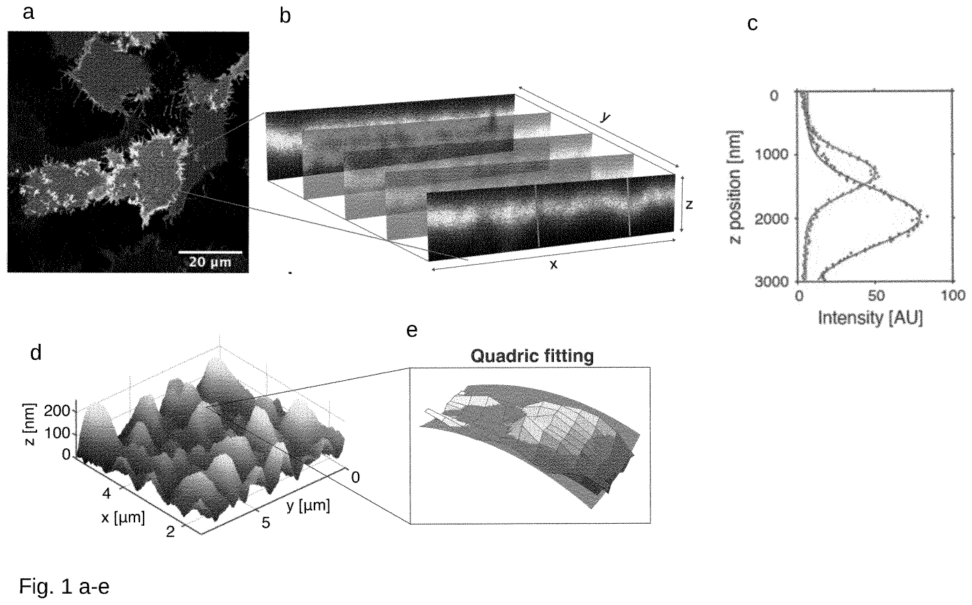

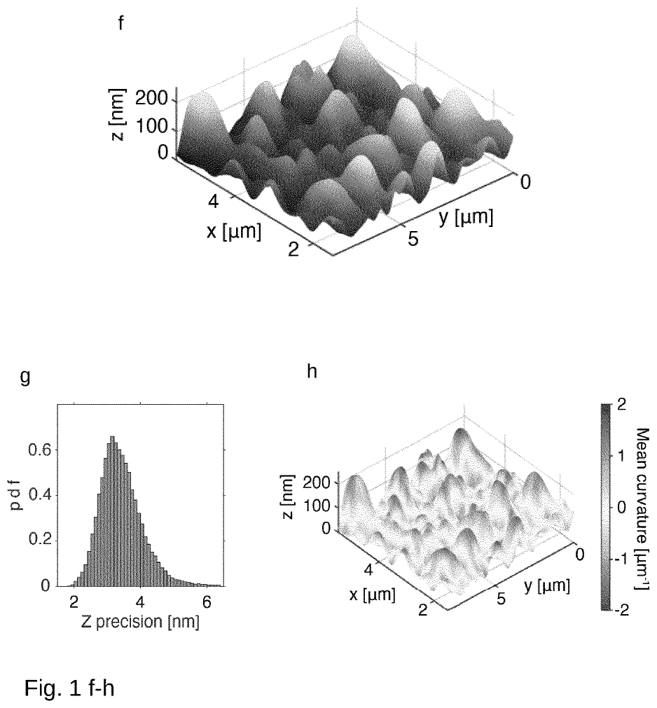

The 3D imaging method obtains the Z position of the adherent part of the plasma membrane of living cells with high accuracy (FIG. 1). We image the plasma membrane in three dimensions by XZY stacks with a voxel size of 30 nm. Examples are shown in FIGS. 1a and 1b. For each XY-pixel we extract an intensity profile in Z, which is fitted with a Gaussian function (Example in FIG. 1c). Here, the fit to the data provides a good estimation of the Z position of the membrane.

Hereafter, a topography map of the surface is generated from the Z positions directly obtained by the Gaussian fits, as seen at an example in FIG. 1d. To improve the localization accuracy, a denoising approach was deployed that removes the high-frequency noise, while maintaining fine spatial fluctuations in a supervised manner. The topography map was treated as a noisy point cloud and error weighted quartic fits are used to retrieve a high accuracy estimate of the Z position and principal curvatures of each pixel4. The surface is fitted pixelwise with a quartic fit (Eq. 1, see Methods) with a window size that is related to the diffraction limit in XY. As a result, a denoised surface was recovered with a mean accuracy in Z of 3.1±1 nm, which reflect the errors associated with the pixelwise estimation of the Z position.

Next, the mean and Gaussian curvature of the recovered surface is calculated using their analytical expressions (Eq. 2 and 3, see Methods). Using the mean and Gaussian curvatures, also the two principal curvatures (Eq. 4, see Methods) were calculated. The two principal curvatures give a measure of the maximum and minimum bending of each point and represent the overall geometry of a point.

### Reconstructing High-Accuracy Topography Maps 3D Imaging

3D membrane topography was reconstructed by a software implemented method. Briefly, XY-slices were smoothened with a mean filter of 3×3 pixels and every XY-position was fitted with a Gaussian in the Z-direction (FIG. 1c). The Z position of the peak of the Gaussian fit localized the Z-position of the plasma membrane (FIG. 1c, ref. 5). The amplitude, i.e. maximum intensity, of the Gaussian fit is proportional to the density of the protein in each pixel.

Poor fits were filtered out using the error metrics from the Gaussian fits based on R2 and uncertainties of the Z position and maximum intensity. Additionally, to remove non-diffraction limited membrane structures, fitted data where the full width at half maximum (FWHM) of the Gaussian significantly exceeds the diffraction limit in Z was removed.

Using dx=dy=30 nm as pixel size in this example, quartic fits were used to denoise the surface extracted by the Z position of Gaussian fits. Each pixel, surrounded by a neighboring pixel window related to the resolution in XY, was fitted to Eq. 1:

$$f ( x , y ) = a 1 \cdot {x}^{2} + a 2 \cdot x \cdot y + a 3 \cdot {y}^{2} + a 4 \cdot x + a 5 \cdot y + a 6 \qquad ( 1 )$$

where a1-a6 are constants. A major advantage of fitting the surface to Eq. 1, is that it provides the ability to obtain an analytical expression for the mean (Eq. 2) and the Gaussian curvature (Eq. 3) of each pixel, H and K respectively.

$$H = ( ( 1 + {f}_{y}^{2} ) \cdot {f}_{{xx}^{-}} 2 \cdot {f}_{x} \cdot {f}_{y} \cdot {f}_{xy} + ( 1 + {f}_{x}^{2} ) {f}_{yy} ) / ( 2 \cdot {( 1 + {f}_{x}^{2} + {f}_{y}^{2} )}^{3 / 2} ) \qquad ( 2 )$$

$$K = ( {f}_{xx} \cdot {f}_{yy} - {f}_{xy}^{2} ) / {( 1 + {f}_{x}^{2} + {f}_{y}^{2} )}^{2} \qquad ( 3 )$$

Here, the functions are defined as first and second order derivatives of Eq. 1:

$${f}_{x} = 2 \cdot a 1 x + a 2 y + a 4 , {f}_{y} = a 2 x + 2 \cdot a 3 y + a 5 , {f}_{xx} = 2 \cdot a 1 , {f}_{yy} = 2 \cdot a 3 , and {f}_{xy} = a 2.$$

The quartic fit of each pixel is error weighted by the error associated with the determination of the Z position from the Gaussian profile fits. The error weighted mean average of the quartic fitted surfaces for a 3×3 grid is taken for the Z position, mean and Gaussian curvatures. By using dx=dy=30 nm, the 3×3 pixels will correspond to an area of 90×90 nm, which is a factor of two below the resolution limit. These 9 pixels can be considered as an independent technical repeat measurement, thus an error weighted standard error of the mean can be used for estimating the accuracy of the Z position. This results in a high precision topography map of the adherent cell membrane of a living cell.

Recovering Receptor Density from Gaussian Fits

This approach makes use of Gaussian fits to recover the position of the membrane in Z (Z-location of the peak of the Gaussian curve, see previous section) and the density of protein (the maximum intensity of the Gaussian curve). The maximum intensity of the Gaussian profile depends on the total amount of labelled receptors and the membrane area that is passing through the confocal volume of the point we are sampling. The latter will vary depending on the angle of the membrane that crosses the confocal volume. This example normalizes for this variation in membrane angles by dividing the maximum intensity of the Gaussian fit by the membrane area crossing the sampled confocal volume. This results in the most accurate representation of receptor density on the recovered topography maps.

### Filtering Criteria for Gaussian Fits

Several filtering criteria were imposed to improve the accuracy of the recovered topography surface and protein density. First, fits with an adjusted R-squared below 0.9 are removed from further analysis. Second, the standard error of the fit for the maximum intensity of the Gaussian must be smaller than 30% of the value of maximum intensity. Third, the standard error of the fit for the Z position should be smaller than 100 nm. Fourth, we filter out Gaussian fits with full width at half maxima (FWHM) larger than 800 nm and smaller than 600 nm. This filter allows us to remove any membrane features that are larger than the axial diffraction limit and do not correspond to a simple membrane bilayer. Collectively, the above filtering accepts typically ^{~}80% of gaussian fits.

### Recovering Ligand Density

Density of fluorescently labelled objects, such as ligands, antibodies has been calculated by averaging along the optical axis the intensity of the ligand channel measured 4 pixels above and below the recovered Z position. This density has been corrected for background signal.

### Example 1

### Cell Lines

Human embryonic kidney (HEK293) cells (ATCC®CRL-1573™) were cultured in DMEM supplemented with 10% FBS. Cell lines were tested routinely for mycoplasma by Eurofins Genomics Mycoplasmacheck. All cell lines were grown at 37° C., 5% CO2, in an atmosphere with 100% humidity.

### Cell Transfection

All cell lines were grown in 8-well Ibidi® chambers with glass bottoms. For each well, a solution of plasmid, Lipofectamine™ LTX Reagent with PLUS™ was made according to manufacturers' protocol in the ratio of 1:3:1, and OptiMEM was added to a final volume of 25 μL. The amount of plasmid used for each well was 0.25 μg β1AR-SNAP, 0.25 μg β2AR-SNAP and 0.25 μg GLP1R-SNAP. After transfection, the cells were left to grow for about 16 hours before imaging.

Activation of β1AR, β2AR and GLP1R is measured by recruitment of miniGₛ, an engineered version of G protein [symbol] s. Similarly, activation of β1AR and β2AR can be probed by nanobody 80 (Nb80), which specifically recognizes the active conformation of β1AR and β2AR. As described above, for each well, a solution of plasmid, Lipofectamine™ LTX Reagent with PLUS™ was made according to manufacturers' protocol in the ratio of 1:3:1, and OptiMEM was added to a final volume of 25 μL. The amount of plasmid used for each well was 0.25 μg β1AR-SNAP and 0.188 μg eGFP-miniGs or 0.188 μg HALO-miniGs or 0.188 μg mRuby2-miniGs or 0.188 μg of eGFP-Nb80, 0.25 μg β2AR-SNAP and 0.188 μg eGFP-miniGs or 0.188 μg of eGFP-Nb80, and 0.25 μg GLP1R-SNAP and 0.188 μg HALO-miniGs. After transfection, the cells were left to grow for about 16 hours before imaging.

### Ligands

Activation of β1AR was measured for the ligands: isoproterenol hydrochloride, BI-167107 and BI-PEG-KK114, epinephrine hydrochloride, norepinephrine bitartrate salt, salbutamol hemisulfate salt, dobutamine hydrochloride, procaterol hydrochloride, CGP20712A methane sulfonate salt, atenolol, carvedilol and carazolol-KK114. Activation of GLP1R was measured for the ligands: [Leu14,Leu28]-Exendin4-Alexa488, [Aib8]-GLP-1(7-37)-Alexa488 and Exendin4[9-39]-Alexa488.

### Description of the Experiments and Measurement Principle

### Live-Cell Protein Labelling and Receptor Activation

To selectively label PM GPCRs we took the following measures: 1) we tagged receptors on the extracellular N terminus and selectively labelled PM GPCRs using cell-impermeable SNAP technology1, 2) we imaged within ^{~}10 min from fluorescent labelling and in the absence of agonists, to minimize the chance of constitutive and ligand mediated internalization2, 3) we validated the method with a prototypic GPCR that is known to reside mostly in the PM, the beta1 adrenergic receptor (β1AR).

Prior to imaging, SNAP-tagged β1AR, β2AR, GLP1R were labelled with SNAP649 according to manufacturers' protocol. Briefly, the cell medium was removed from each well and 100 μL of new medium premixed with 0.5 μL of a 50 nmol/μL solution of SNAP-Surface® was added to the cells and the labelling reaction proceeded for 10 min at 37° C. Next, the medium was replaced with 200 μL of Leibovitz's medium and the sample was washed 3 times before imaging.

### Results

FIG. 1 shows the generation of high-accuracy topography maps of plasma membranes of living cells. FIG. 1a shows a fluorescence confocal image of HEK293 cells over-expressing a SNAP construct of the β1AR receptor labeled with SNAPSurface649. FIG. 2b illustrates an XZY-stack (dz/dx/dy=30 nm) of the highlighted area in FIG. 1a. FIG. 1c shown extracted intensity Z-profiles of the blue and red linescans highlighted in FIG. 1b and their corresponding Gaussian fits overlaid. The axial position of the Gaussian peak corresponds to the Z position of the membrane, whereas the amplitude of the Gaussian peak is proportional to protein density. FIG. 1d is a topography map of the area shown in FIG. 1b reconstructed from the Z positions obtained from the Gaussian fitting. FIG. 1e is a local error weighted quartic fit for a 3×3 pixel, 90 nm×90 nm, area using Eq. 1 (see Methods). FIG. 1f is a recovered denoised topography map after quartic fitting (same area as in FIG. 1d). FIG. 1g shows the localization precision of the Z positions calculated as the error weighed standard error of the mean for a 3×3 pixel, 90 nm×90 nm, area of the denoised topography maps. Because this 90×90 nm area is a factor of two below the resolution limit we consider these 9 pixels as an independent technical repeat measurement. FIG. 1f is a topography map from FIG. 1f overlaid with mean membrane curvature.

FIG. 2 shows examples of how receptor density and activation is organized into domains at the plasma membrane. FIG. 2a is 2D projection of normalized receptor density (β1AR). FIG. 2b is a 2D projection of Nb80 density, a typical conformational probe used for the β1AR and β2AR receptor. FIG. 2c shows a 2D projection of fluorescent ligand density, for BI-PEG-KK114. FIG. 2d shows a 2D projection of ratio Nb80/β1AR, i.e. conformational probe to receptor, reveals spatial variations in activation probability. FIG. 2e shows a 2D projection for ratio Nb80/BI-PEG-KK114, i.e. conformational probe to ligand, reveals spatial variations in intrinsic efficacy. FIG. 2f shows a schematic of domain-based data analysis shows domains of high ratio above a defined threshold. Outlined are isolated domains that may form a group or classification class. This allows us extract 3 ligand specific parameters: 1) the area of a domain, 2) the ratio in a domain and 3) the number of domains produced by a ligand.

FIG. 3 shows exemplary a domain-based characterization of different ligands and different concentrations of the same ligand. FIG. 3a is a scatter plot of the intrinsic efficacy of domains versus log (EC₅₀) for 8 different ligands. Data points in green are full agonists and partial agonists are shown in purple. Errorbars in y represent standard error of the mean and in x represent the errors from ref.⁶. FIG. 3b is the ratio of intrinsic efficacy in domains over intrinsic efficacy in background plotted against concentration of isoproterenol. Data points are shown in blue and red dashed line is a fit.

FIG. 4 shows as an example of the method that conformational states of GLP1R are segregated by plasma membrane curvature. FIG. 4a is a curvatures space plot (C1 vs. C2) of relative GLP1R density in the absence of ligand. FIGS. 4b to 4d are curvature space plots (C1 vs. C2) of relative GLP1R density (left panel), relative ligand/receptor ratio (middle panel) and relative miniGs/receptor (right panel) for the agonists Glp-1 (FIG. 4b) and Exendin-4 (FIG. 4c) and antagonist Exendin-4 (9-39) (FIG. 4d).

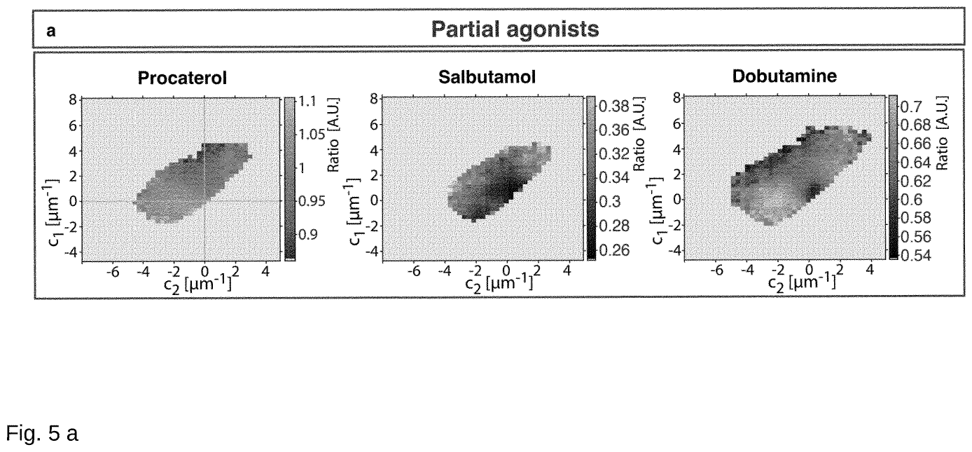

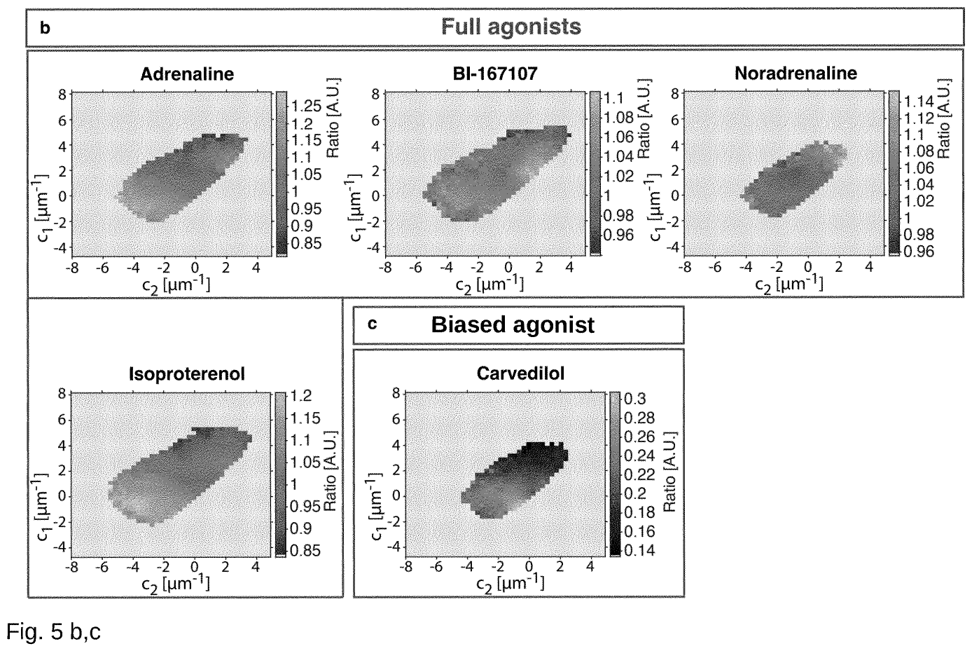

FIG. 5 shows an example results where different β1AR ligands give rise to different patterns of activation in curvature space. FIGS. 5a to c are curvature space plots (C1 vs. C2) for Nb80/β1AR ratio for 3 partial agonists (FIG. 5a), 4 full agonists (FIG. 5b) and 1 biased agonist (FIG. 5c).

### Example 2

As long as not explicitly stated herein, the methods and experiments correspond to the other examples above.

### Deconvolution of State Occupancy and Reactivity.

The deconvolution of state occupancy and reactivity relies on segregated spatial patterns of intrinsic activation probability (PIA) (FIG. 6b). In combination with the underlying histogram of activation probabilities, this allowed us to identify four activity states: the inactive states S1 and S2 and the active states S3 and S4. The occupancy of the S1 state directly corresponds to the number of pixels with PIA=0, normalized by the total number of pixels. To disentangle the occupancy of the S2, S3 and S4 state, we first use a Gaussian mixture model with two components to fit the histogram of PIA (i.e., population F2 and F3 in FIG. 6d). For each Gaussian this results in the average PIA of the population, termed Mx, and the relative weight of the population, termed wx. Furthermore, for each population we extract the average miniG density, termed miniGx. Here, the subscript x is used as a substitute that refers either to calculations for active state S3 or S4.

### Results

The present disclosure of this example relies on simultaneous imaging of β1AR and miniGs, a widely used Gas surrogate, with two spectrally distinct fluorophores in live cells (FIG. 6a). MiniGs binds reversibly to activated GPCRs with minimal functional perturbation and has been shown to accurately report basal and ligand-dependent activation. To obtain a direct measure of the intrinsic activation probability of GPCRs at the PM, we performed a ratiometric analysis of miniGs and β1AR densities, using a previously developed high-content image analysis method.

At the basal state, the activity patterns are composed of three spatially segregated populations with distinct activation probabilities: PIA=0 (F1), a low PIA (F2) and a high PIA (F3) population (FIG. 6c). A histogram of PIA reveals a trimodal distribution, consisting of an inactive population F1 and a mixture of populations F2 and F3 (FIG. 6d). All receptors in the F1 population appear to be ‘locked’ in an inactive conformation that within our detection limit is not spontaneously (i.e., in the absence of agonist) interconverting to an active conformation.

After assigning the different β1AR conformers in the F2/F3 populations, the histogram (in FIG. 6d) reveals an evident heavy-tailed Gaussian distribution that comprises the F2 and F3 populations. Using a Gaussian mixture model with two components, the relative weight of each population and its average PIA is determined. This corresponds to 18% and 25% for the F2 and F3 populations respectively (black arrow heads, FIG. 6d). Because this is well below 100%, it suggests that, at basal state, receptors in the F2/F3 populations occupy most of their time an inactive conformation.

Precise knowledge of PIA and the relative abundance of the F1, F2 and F3 populations allows to directly quantify in living cells the occupancy of each receptor state and the reactivity of the active states. This is achieved by solving a set of linear equations that describe the F2 and F3 populations in terms of their average activation probability, relative occurrence and total miniGs recruitment. To deconvolve the state occupancies and reactivities, this high-content analysis relies on the following three fundamental considerations:

1, The average activation probability of a population (black triangles above F2 and F3, FIG. 6d) is equal to the number of receptors in the active state divided by the total number of receptors in the population. 2, The area under the histogram of a population (F2 or F3) represents the sum of all active and inactive receptors in the population. 3, The reactivity of a state, i.e., its affinity for binding miniGs, is calculated as the amount of miniGs recruited by a state divided by the number of receptors that populate that state.

This framework provides a quantification of state occupancy at the level of a single, living cell. For example, under basal conditions the inactive states are populated by 77% S1 and 18% S2 receptors, whereas a minor fraction of receptors consists of the active S3 (3%) and S4 (2%) states (FIG. 6e). Next to the identification of the four canonical states, the spatial segregation of activity populations can also be leveraged to understand the sequence of interconversion during GPCR activation. We find that the inactive S1 state can interconvert to the more pliable inactive S2 state. Subsequently, the S2 state can spontaneously sample either the S3 or the S4 state (FIG. 6f)

FIG. 7 shows the Redistribution of activation probabilities and states by full and inverse agonists. FIG. 7a is a histogram of activation probability of a single representative cell for increasing concentration of full agonist ISO starting from the basal conditions (APO).

FIGS. 7b and c show distributions of occupancy for the inactive states (7b), S1 and S2, and for the active states (7c), S3 and S4, for increasing ISO concentrations. This validates the ability of our method to reveal titratable changes in occupancy with a full agonist. FIG. 7d is a histogram of activation probability of a single representative cell for APO, 9 nM ISO and 70 nM CGP after incubation for 15- and 45-min. FIGS. 7e and f, show distributions of occupancy for the inactive states (7e), S1 and S2, and for the active states (7f), S3 and S4, for full and inverse agonists. These results validate the detection of reversible changes in state occupancy. Full agonist data is representative for nC=9 and nR=2, inverse agonist data nC=22 and nR=2.

The methods and systems have been tested with different ligands. The methods and systems can be used to study spatial patterns of receptor activation for any receptor and any ligand at any ligand concentration. It is evident that the principles described are not limited to ligand-receptor interactions but that any other conformational change in a membrane bound molecule can be detected and characterized. The conformation change may be triggered by ligand interaction or any other change in the environment of the membrane bound molecule.

1. A method for characterizing a distribution of states of a membrane bound molecule, the method comprising:
determining a plurality of spatially resolved membrane properties of a biological membrane,
determining, for a plurality of locations, a spatially resolved membrane molecule signal related to the membrane bound molecule at the plurality of locations,
grouping the spatially resolved membrane molecule signals to classes of at least one of the membrane properties or state classes of the membrane bound molecules,
identifying the distribution of states of the membrane bound molecule based on the distribution of the classes.

2. The method of claim 1, further comprising normalizing the spatially resolved membrane molecule signal by the spatially resolved membrane property.

3. The method of claim 1 or 2, wherein identifying the distribution of states comprises deconvoluting state occupancies of different states of the membrane bound molecule.

4. The method of any one of the preceding claims, further comprising obtaining patterns of the distribution of conformational states of the membrane bound molecules.

5. The method of any one of the preceding claims, further comprising inducing a conformational change to the membrane bound molecule.

6. The method of claim 5, wherein the conformational change to the membrane bound molecule comprises addition of at least one of a ligand and an antibody.

7. The method of any one of the preceding claims, wherein the determining the spatially resolved membrane molecule signal comprises detecting a signal of at least one of a tag attached to the membrane molecule, an antibody to the membrane bound molecule, a conformational biosensor, a ligand to the membrane bound molecule.

8. The method of any one of the preceding claims, wherein the determining the spatially resolved membrane molecule signal comprises determining at least one of a ratio of signals of an antibody to membrane bound molecule, ligand to antibody, ligand to membrane bound molecule, a conformational biosensor to membrane bound molecule, antibody to conformational biosensor, and ligand to conformational biosensor.

9. The method of any one of the preceding claims, wherein the determining the spatially resolved membrane molecule signal comprises determining a signal of the membrane bound molecule and further comprising determining an additional spatially resolved molecules signal relating to at least one of a ligand, an antibody and a conformational biosensor.

10. The method of any one of the preceding claims, wherein the determining the spatially resolved membrane molecule signal comprises determining, for the plurality of locations, a state specific spatially resolved membrane molecule signal relating to a conformational state of the membrane bound molecule.

11. The method of any one of the preceding claims, wherein the biological membrane is a plasma membrane of a biological cell.

12. The method of any one of the preceding claims, wherein the membrane molecule is a membrane bound receptor protein.

13. The method of any one of the preceding claims, wherein the plurality of spatially resolved membrane properties are topography parameters of the membrane.

14. A method for characterizing a state of a membrane bound molecule, the method comprising:
determining a plurality of spatially resolved membrane properties of a biological membrane,
determining, for a plurality of locations, a spatially resolved membrane molecule signal related to the membrane bound molecule at the plurality of locations,
grouping the spatially resolved membrane molecule signals to classes of the membrane properties to obtain a state specific membrane molecule pattern,
comparing the state specific membrane molecule pattern with patterns in a database and identifying a conformational state of the membrane bound molecule.

15. A method for characterizing a membrane bound molecule, the method comprising:
determining a plurality of spatially resolved membrane properties of a biological membrane,
determining, for a plurality of locations, a spatially resolved membrane molecule signal related to the membrane bound molecule at the plurality of locations,
grouping the spatially resolved membrane molecule signals to classes of the membrane properties to obtain a first state specific membrane molecule pattern,
inducing a conformational change to the membrane bound molecule, and
monitoring a change in the grouping of the spatially resolved receptor signals and obtaining a second state specific membrane molecule pattern.

16. The method of claim 15, further comprising storing the second state specific membrane molecule pattern in a database representative for the conformational state induced by the conformational change.

17. The method of claim 15 or 16, wherein inducing a conformational change to the membrane bound molecule comprises addition of at least one of a ligand and an antibody.

18. The method of any one of claims 15 to 17, wherein the determining the spatially resolved membrane molecule signal comprises detecting a signal of at least one of a tag attached to the membrane molecule, an antibody to the membrane bound molecule, a ligand to the membrane bound molecule.

19. The method of any one of claims 15 to 18, wherein the determining the spatially resolved membrane molecule signal comprises determining at least one of a ratio of signals of an antibody to membrane bound molecule, ligand to antibody and ligand to membrane bound molecule.

20. The method of any one of claims 15 to 19, wherein the biological membrane is a plasma membrane of a biological cell.

21. The method of any one of claims 15 to 20, wherein the membrane molecule is a membrane bound receptor protein.

22. The method of any one of claims 15 to 21, wherein the plurality of spatially resolved membrane properties are topography parameters of the membrane.

23. The method of any one of claims 15 to 22, wherein the plurality of spatially resolved membrane properties comprise at least one membrane curvature (C1, C2).

24. The method of any one of claims 15 to 23, wherein determining the plurality of spatially resolved membrane properties comprises fluorescence imaging.

25. The method of any one of claims 15 to 24, wherein the determining, for a plurality of locations, the spatially resolved membrane molecule signal, comprises fluorescence imaging of fluorescent tags attached to at least one of the membrane molecule, a ligand to membrane molecule, and an antibody to the membrane molecule.

26. An apparatus for characterizing a membrane bound molecule, the apparatus comprising a fluorescence imaging device for imaging fluorescence in and at cell membranes and a data evaluation, the data evaluation implementing the methods of any of the preceding claims.

27. A data storage comprising data representative for a state specific membrane molecule pattern in a membrane property spaces and representative for the conformational state of the membrane molecule.
